SEQUENCE LISTING (72 sequences)

SEQ ID NO: 1 - DNA, 3701 bp, synthetic construct
  source 1..3701: mol_type = other DNA; note = Linearized vector pKBXInH5
taatgtgcctgtcaaatggacgaagcagggattctgcaaaccctatgctactccgtcaagccgtcaattgtctgattcgttaccaattatgacaacttgacggctacatcattcactttttcttcacaaccggcacggaactcgctcgggctggccccggtgcattttttaaatacccgcgagaaatagagttgatcgtcaaaaccaacattgcgaccgacggtggcgataggcatccgggtggtgctcaaaagcagcttcgcctggctgatacgttggtcctcgcgccagcttaagacgctaatccctaactgctggcggaaaagatgtgacagacgcgacggcgacaagcaaacatgctgtgcgacgctggcgatatcaaaattgctgtctgccaggtgatcgctgatgtactgacaagcctcgcgtacccgattatccatcggtggatggagcgactcgttaatcgcttccatgcgccgcagtaacaattgctcaagcagatttatcgccagcagctccgaatagcgcccttccccttgcccggcgttaatgatttgcccaaacaggtcgctgaaatgcggctggtgcgcttcatccgggcgaaagaaccccgtattggcaaatattgacggccagttaagccattcatgccagtaggcgcgcggacgaaagtaaacccactggtgataccattcgcgagcctccggatgacgaccgtagtgatgaatctctcctggcgggaacagcaaaatatcacccggtcggcaaacaaattctcgtccctgatttttcaccaccccctgaccgcgaatggtgagattgagaatataacctttcattcccagcggtcggtcgataaaaaaatcgagataaccgttggcctcaatcggcgttaaacccgccaccagatgggcattaaacgagtatcccggcagcaggggatcattttgcgcttcagccatacttttcatactcccgccattcagagaagaaaccaattgtccatattgcatcagacattgccgtcactgcgtcttttactggctcttctcgctaaccaaaccggtaaccccgcttattaaaagcattctgtaacaaagcgggaccaaagccatgacaaaaacgcgtaacaaaagtgtctataatcacggcagaaaagtccacattgattatttgcacggcgtcacactttgctatgccatagcatttttatccataagattagcggatcctacctgacgctttttatcgcaactctctactgtttctccatacccgtttttttggcatatggagctcccatggtgtacacctaggagatctgcgatcgcgtttggaggtaataaatggcagtgcaacactctaatgcgcctctgatcgattgtggagccgaaatgaaaaaacagcataaggaggccgcgcctgaaggtgctgcacctgctcaagggaaagctcctgcggctgaagcgaaaaaagaagaagcgcccaaacccaaaccggtcgtctctagtgggatcgaggaaaacctttatttccagtctcatcatcatcaccaccacggctctcagtctggcggannnnnnnnnnnntgatagtcggctgcaactttatccgcctgtgactagtgctagcggcgcgccctcgagggtaccgaattcgcggccgccggtctgataaaacagaatttgcctggcggcagtagcgcggtggtcccacctgaccccatgccgaactcagaagtgaaacgccgtagcgccgatggtagtgtgggttctccccatgcgagagtagggaactgccaggcatcaaataaaacgaaaggctcagtcgaaagactgggcctttcgttttatctgttgtttgtcggtgaacgctctcctgagtaggacaaatccgccgggagcggatttgaacgttgcgaagcaacggcccggagggtggcgggcaggacgcccgccataaactgccaggcatcaaattaagcagaaggccatcctgacggatggcctttttgcgtttctacaaactcttttgtttatttttctaaatacattcaaatatgtatccgctcatgagacaataaccctgataaatgcttcaataatattgaaaaaggaagagtatgagccatattcaacgggaaacgtcttgctcgaggccgcgattaaattccaacatggatgctgatttatatgggtataaatgggctcgcgataatgtcgggcaatcaggtgcgacaatctatcgattgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccgggaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataagcttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaataaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatg

SEQ ID NO: 2 - DNA, 741 bp, synthetic construct
  source 1..741: mol_type = other DNA; note = Insert sequence of Epidermal Growth Factor (eGFP)
cagtctggcggaatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtgatagtcggct

SEQ ID NO: 3 - DNA, 2328 bp, synthetic construct
  source 1..2328: mol_type = other DNA; note = pKLpositive vector
caggtggcacttttcggggaaatgtgcgcggaacccctatttgtttatttttctaaatacattcaaatatgtatccgctcatgagacaataaccctgataaatgcttcaataatattgaaaaaggaagagtatgagccatattcaacgggaaacgtcttgctcgaggccgcgattaaattccaacatggatgctgatttatatgggtataaatgggctcgcgataatgtcgggcaatcaggtgcgacaatctatcgattgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccgggaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataagcttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaataaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgcccatatggagctcccatggtgtacacctaggagatctgcgatcgcgtttggaggtaaataaatgggtcatcaccatcatcatcacggaagcggttctagcggtatggtgagcagtggcgaagatattttctcgggcttggttccgattctgatcgagctggagggcgatgtgaacggtcatcgttttagcgttcgcggtgaaggttatggcgacgcgagcaacggcaaactggaaattaagttcatctgcacgannnnnnnnnnnnacgaccaatagcgtgctgagcaaagatccgcaggaacgccgtgatcacatggtcctggtggaatttgtgaccgctgcgggcttgagcctgggtatggacgagctgtataagagctaagtgactagtgctgtgactagtgctagcggcgcgccctcgagggtaccgaattcgcggccgc

SEQ ID NO: 4 - DNA, 483 bp, synthetic construct
  source 1..483: mol_type = other DNA; note = Remaining fuGFP as 483 bp insert
gttcatctgcacgaccggtcgcctgccggtgccttggccgaccttggtgacgaccttgtcgtatggcgtgcagtgttttgcgaagtatccggagcacatgcgccaaaacgatttctttaaaagtgcgatgccggacggttacgtccaggagcgtaccatttccttcaaggaagatggcacgtacaaaactcgcgcagaggttaagtttgaaggtgaagcgctggtcaatcgtatcgatttgaagggtttggagtttaaagaggatggtaacattctgggccataaactggagtatagcttcaacagccattatgtttacattacggcagacaagaatcgtaacggcttggaggcccaattccgtattcgccacaatgttgatgacggtagcgtccaactggccgaccattaccaacagaacaccccaattggtgagggtccggtgttgctgccggaacaacactatctgacgaccaatagcgt

SEQ ID NO: 5 - DNA, 2414 bp, synthetic construct
  source 1..2414: mol_type = other DNA; note = pKLShv
caggtggcacttttcggggaaatgtgcgcggaacccctatttgtttatttttctaaatacattcaaatatgtatccgctcatgagacaataaccctgataaatgcttcaataatattgaaaaaggaagagtatgagccatattcaacgggaaacgtcttgctcgaggccgcgattaaattccaacatggatgctgatttatatgggtataaatgggctcgcgataatgtcgggcaatcaggtgcgacaatctatcgattgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccgggaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataagcttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaataaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgcccatatggagctcccatggtgtacacctaggagatctgcgatcgcgtttgcgatcgcgtttggaggtaaataaatgggtcatcaccatcatcatcacggaagcggttctagcggtatggtcagtaaaggtgaggagttgtttactggtgtcgttcctattcttgtcgagttggacggggatgtgaacgggcacaagttttcggtacgtggcgagggtgaaggggatgcaactaatggcaagttgacgttgaagtttatatgcacgactgggaagctcccagtgccatggccaactttggtgactaccttannnnnnnnnnnncactatctttctacacaaagcctgctttctaaggacccaaatgagaaacgcgaccacatggtcttgcttgagttcgtgacagcggcagggattaccttgggcatggacgagctctacaaggggatcgaggaaaacctgtacttctaagtgactagtgctgtgactagtgctagcggcgcgccctcgagggtaccgaattcgcggccgc

SEQ ID NO: 6 - DNA, 402 bp, synthetic construct
  source 1..402: mol_type = other DNA; note = Remaining Superfolder Green Fluorescent Protein (sfGFP) as 48bp insert
acctacggtgtacaatgcttctcgcgttaccccgatcacatgaagcaacacgatttcttcaagtcagcaatgcctgaaggttacgtccaagaacgtactatatcattcaaagacgacggtacctacaagactcgggcggaagttaagttcgaaggtgacactttagtcaatcgtatcgagttaaagggtatcgatttcaaagaggatggcaacattttaggacacaagctggagtacaactttaacagccacaatgtatacattactgccgacaagcaaaagaacggcatcaaggcaaatttcaagattagacataacgtcgaagacggctccgtgcaattagcagatcattatcaacagaacacgccgatcggcgacggccccgtgttattacccgacaat

SEQ ID NO: 7 - DNA, 12 bp, synthetic construct
  source 1..12: mol_type = other DNA
cagtctggcgga

SEQ ID NO: 8 - DNA, 12 bp, synthetic construct
  source 1..12: mol_type = other DNA
tgatagtcggct

SEQ ID NO: 9 - DNA, 14 bp, synthetic construct
  source 1..14: mol_type = other DNA
gttcatctgcacga

SEQ ID NO: 10 - DNA, 14 bp, synthetic construct
  source 1..14: mol_type = other DNA
acgaccaatagcgt

SEQ ID NO: 11 - DNA, 10 bp, synthetic construct
  source 1..10: mol_type = other DNA
gactacctta

SEQ ID NO: 12 - DNA, 10 bp, synthetic construct
  source 1..10: mol_type = other DNA
cactatcttt

SEQ ID NO: 13 - DNA, 11 bp, synthetic construct
  source 1..11: mol_type = other DNA
tgactacctta

SEQ ID NO: 14 - DNA, 11 bp, synthetic construct
  source 1..11: mol_type = other DNA
cactatctttc

SEQ ID NO: 15 - DNA, 12 bp, synthetic construct
  source 1..12: mol_type = other DNA
gtgactacctta

SEQ ID NO: 16 - DNA, 12 bp, synthetic construct
  source 1..12: mol_type = other DNA
cactatctttct

SEQ ID NO: 17 - DNA, 13 bp, synthetic construct
  source 1..13: mol_type = other DNA
ggtgactacctta

SEQ ID NO: 18 - DNA, 13 bp, synthetic construct
  source 1..13: mol_type = other DNA
cactatctttcta

SEQ ID NO: 19 - DNA, 14 bp, synthetic construct
  source 1..14: mol_type = other DNA
tggtgactacctta

SEQ ID NO: 20 - DNA, 14 bp, synthetic construct
  source 1..14: mol_type = other DNA
cactatctttctac

SEQ ID NO: 21 - DNA, 15 bp, synthetic construct
  source 1..15: mol_type = other DNA
ttggtgactacctta

SEQ ID NO: 22 - DNA, 15 bp, synthetic construct
  source 1..15: mol_type = other DNA
cactatctttctaca

SEQ ID NO: 23 - DNA, 16 bp, synthetic construct
  source 1..16: mol_type = other DNA
tttggtgactacctta

SEQ ID NO: 24 - DNA, 15 bp, synthetic construct
  source 1..15: mol_type = other DNA
cactatctttctaca

SEQ ID NO: 25 - DNA, 17 bp, synthetic construct
  source 1..17: mol_type = other DNA
ctttggtgactacctta

SEQ ID NO: 26 - DNA, 15 bp, synthetic construct
  source 1..15: mol_type = other DNA
cactatctttctaca

SEQ ID NO: 27 - DNA, 17 bp, synthetic construct
  source 1..17: mol_type = other DNA
actttggtgactacctt

SEQ ID NO: 28 - DNA, 15 bp, synthetic construct
  source 1..15: mol_type = other DNA
cactatctttctaca

SEQ ID NO: 29 - DNA, 17 bp, synthetic construct
  source 1..17: mol_type = other DNA
aactttggtgactacct

SEQ ID NO: 30 - DNA, 15 bp, synthetic construct
  source 1..15: mol_type = other DNA
cactatctttctaca

SEQ ID NO: 31 - DNA, 17 bp, synthetic construct
  source 1..17: mol_type = other DNA
caactttggtgactacc

SEQ ID NO: 32 - DNA, 20 bp, synthetic construct
  source 1..20: mol_type = other DNA
cactatctttctacacaaag

SEQ ID NO: 33 - DNA, 21 bp, synthetic construct
  source 1..21: mol_type = other DNA
ccaactttggtgactacctta

SEQ ID NO: 34 - DNA, 21 bp, synthetic construct
  source 1..21: mol_type = other DNA
cactatctttctacacaaagc

SEQ ID NO: 35 - DNA, 22 bp, synthetic construct
  source 1..22: mol_type = other DNA
gccaactttggtgactacctta

SEQ ID NO: 36 - DNA, 22 bp, synthetic construct
  source 1..22: mol_type = other DNA
cactatctttctacacaaagcc

SEQ ID NO: 37 - DNA, 23 bp, synthetic construct
  source 1..23: mol_type = other DNA
ggccaactttggtgactacctta

SEQ ID NO: 38 - DNA, 23 bp, synthetic construct
  source 1..23: mol_type = other DNA
cactatctttctacacaaagcct

SEQ ID NO: 39 - DNA, 24 bp, synthetic construct
  source 1..24: mol_type = other DNA
tggccaactttggtgactacctta

SEQ ID NO: 40 - DNA, 24 bp, synthetic construct
  source 1..24: mol_type = other DNA
cactatctttctacacaaagcctg

SEQ ID NO: 41 - DNA, 25 bp, synthetic construct
  source 1..25: mol_type = other DNA
atggccaactttggtgactacctta

SEQ ID NO: 42 - DNA, 25 bp, synthetic construct
  source 1..25: mol_type = other DNA
cactatctttctacacaaagcctgc

SEQ ID NO: 43 - DNA, 26 bp, synthetic construct
  source 1..26: mol_type = other DNA
catggccaactttggtgactacctta

SEQ ID NO: 44 - DNA, 25 bp, synthetic construct
  source 1..25: mol_type = other DNA
cactatctttctacacaaagcctgc

SEQ ID NO: 45 - DNA, 27 bp, synthetic construct
  source 1..27: mol_type = other DNA
ccatggccaactttggtgactacctta

SEQ ID NO: 46 - DNA, 27 bp, synthetic construct
  source 1..27: mol_type = other DNA
cactatctttctacacaaagcctgctt

SEQ ID NO: 47 - DNA, 28 bp, synthetic construct
  source 1..28: mol_type = other DNA
gccatggccaactttggtgactacctta

SEQ ID NO: 48 - DNA, 28 bp, synthetic construct
  source 1..28: mol_type = other DNA
cactatctttctacacaaagcctgcttt

SEQ ID NO: 49 - DNA, 29 bp, synthetic construct
  source 1..29: mol_type = other DNA
tgccatggccaactttggtgactacctta

SEQ ID NO: 50 - DNA, 29 bp, synthetic construct
  source 1..29: mol_type = other DNA
cactatctttctacacaaagcctgctttc

SEQ ID NO: 51 - DNA, 30 bp, synthetic construct
  source 1..30: mol_type = other DNA
gtgccatggccaactttggtgactacctta

SEQ ID NO: 52 - DNA, 30 bp, synthetic construct
  source 1..30: mol_type = other DNA
cactatctttctacacaaagcctgctttct

SEQ ID NO: 53 - DNA, 31 bp, synthetic construct
  source 1..31: mol_type = other DNA
agtgccatggccaactttggtgactacctta

SEQ ID NO: 54 - DNA, 31 bp, synthetic construct
  source 1..31: mol_type = other DNA
cactatctttctacacaaagcctgctttcta

SEQ ID NO: 55 - DNA, 32 bp, synthetic construct
  source 1..32: mol_type = other DNA
cagtgccatggccaactttggtgactacctta

SEQ ID NO: 56 - DNA, 31 bp, synthetic construct; identical to SEQ ID NO: 54
  source 1..31: mol_type = other DNA

SEQ ID NO: 57 - DNA, 33 bp, synthetic construct
  source 1..33: mol_type = other DNA
ccagtgccatggccaactttggtgactacctta

SEQ ID NO: 58 - DNA, 33 bp, synthetic construct
  source 1..33: mol_type = other DNA
cactatctttctacacaaagcctgctttctaag

SEQ ID NO: 59 - DNA, 34 bp, synthetic construct
  source 1..34: mol_type = other DNA
cccagtgccatggccaactttggtgactacctta

SEQ ID NO: 60 - DNA, 34 bp, synthetic construct
  source 1..34: mol_type = other DNA
cactatctttctacacaaagcctgctttctaagg

SEQ ID NO: 61 - DNA, 35 bp, synthetic construct
  source 1..35: mol_type = other DNA
tcccagtgccatggccaactttggtgactacctta

SEQ ID NO: 62 - DNA, 35 bp, synthetic construct
  source 1..35: mol_type = other DNA
cactatctttctacacaaagcctgctttctaagga

SEQ ID NO: 63 - DNA, 36 bp, synthetic construct
  source 1..36: mol_type = other DNA
ctcccagtgccatggccaactttggtgactacctta

SEQ ID NO: 64 - DNA, 36 bp, synthetic construct
  source 1..36: mol_type = other DNA
cactatctttctacacaaagcctgctttctaaggac

SEQ ID NO: 65 - DNA, 37 bp, synthetic construct
  source 1..37: mol_type = other DNA
gctcccagtgccatggccaactttggtgactacctta

SEQ ID NO: 66 - DNA, 37 bp, synthetic construct
  source 1..37: mol_type = other DNA
cactatctttctacacaaagcctgctttctaaggacc

SEQ ID NO: 67 - DNA, 38 bp, synthetic construct
  source 1..38: mol_type = other DNA
agctcccagtgccatggccaactttggtgactacctta

SEQ ID NO: 68 - DNA, 38 bp, synthetic construct
  source 1..38: mol_type = other DNA
cactatctttctacacaaagcctgctttctaaggaccc

SEQ ID NO: 69 - DNA, 39 bp, synthetic construct
  source 1..39: mol_type = other DNA
aagctcccagtgccatggccaactttggtgactacctta

SEQ ID NO: 70 - DNA, 39 bp, synthetic construct
  source 1..39: mol_type = other DNA
cactatctttctacacaaagcctgctttctaaggaccca

SEQ ID NO: 71 - DNA, 40 bp, synthetic construct
  source 1..40: mol_type = other DNA
gaagctcccagtgccatggccaactttggtgactacctta

SEQ ID NO: 72 - DNA, 40 bp, synthetic construct
  source 1..40: mol_type = other DNA
cactatctttctacacaaagcctgctttctaaggacccaa

(19) United States
(12) Patent Application Publication
(10) Pub. No.: US 2024/0240220 A1
(43) Pub. Date: Jul. 18, 2024

(54) Seamless Cloning Method with Static Recovery Period

(71) Applicant: Zhenyu Zhu, Lynnfield, MA (US)

(72) Inventor: Zhenyu Zhu, Lynnfield, MA (US)

(21) Appl. No.: 18/413,131

(22) Filed: Jan. 16, 2024

Related U.S. Application Data
(60) Provisional application No. 63/439,751, filed on Jan. 18, 2023.

(51) Int. Cl. C12P 21/00; C12N 15/70

(52) U.S. Cl. CPC C12P 21/00; C12N 15/70; C12N 2800/101; C12N 2830/005

(57) ABSTRACT

The invention relates to a seamless cloning method, comprising a single assembly step of two or more polynucleotides, e.g., a plasmid vector and a gene insert, conducted at 58 to 100° C., the preferred temperature(s) being the same or greater than a particular one of the following temperatures: 58° C., 59° C., 60° C., 61° C., 62° C., 63° C., 64° C., 65° C., 66° C., 67° C., 68° C., 69° C., 70° C., 71° C., 72° C., 73° C., 74° C., 75° C., 76° C., 77° C., 78° C., 79° C., 80° C., 84° C., 88° C., 92° C., 96° C. and 100° C., and a transformation into chemically competent cells for covalent linking, preferably including a static recovery period.

### BACKGROUND

Seamless cloning, a recombinant molecular biology technique, enables the insertion of one or more DNA fragments into a vector without reliance on specific sequences and without leaving any undesired scars. The Gibson Assembly (GA) method exemplifies this approach, which can achieve up to a straightforward combination of ten DNA fragments. The method hinges on incorporating homologous regions at the ends of the fragments to be cloned. Subsequently, coordinated actions of an exonuclease that trims 5′ ends to form 3′ compatible overhangs, a DNA polymerase that fills in gaps in annealed fragments, and a DNA ligase that seals the nicks in the assembled DNA facilitate the creation of recombinant DNA.

Several methods/kits based on seamless, sequence homology of DNA assembly, are available commercially or are described in the literature (see references 1-39 below). Seamless, sequence homology based ligase independent methods of DNA assembly, whether from commercially available kits, and/or as described in the literature with and without current product, are summarized in Table 1, listing, alphabetically, the method name, reference, reaction temperature, reaction time, vector amount, and inactivation temperature, if available.

TABLE 1
The comparison of seamless cloning
methods based on homologous sequences.

| | | Assembly | | | Inacti- |
|---|---|---|---|---|---|
| | | Reaction | Shortest | | vation |
| | | temper- | Reaction | Vector | temper- |
| Cloning method | | ature | time | amount | ature |
| name | Ref | ° C. | (min) | (ng) | ° C. |
| AccuRapid | 1 | 50 | 30 | 25 | N/A |
| Cloning Kit | | | | | |
| Choo-Choo | 2 | 0 | 45 | 50 | N/A |
| Cloning Kits | | | | | |
| ClonExpress Entry | 3, | 50 | 5 | 50 | N/A |
| One Step Cloning | 4 | | | | |
| Kit | | | | | |
| Cold fusion | 5 | Room | 5/10 | 10-100 | N/A |
| Cloning | | temperature | | | |
| | | at step 1, | | | |
| | | ice on step2 | | | |
| Complementary | 6 | Room | 3 | 30-50 | 75 |
| annealing mediated | | temperature | | | |
| by exonuclease 1 | | | | | |
| Complementary | 6 | 37 | 5 | 30-50 | 75 |
| annealing mediated | | | | | |
| by exonuclease 2 | | | | | |
| Complementary | 6 | 50 | 30 | 30-50 | N/A |
| annealing mediated | | | | | |
| by exonuclease 3 | | | | | |
| Complementary | 6 | 37 step1, | 30/15 | 30-50 | 75 |
| annealing mediated | | 37 step 2 | | | |
| by exonuclease 4 | | | | | |
| DATEL | 7 | 94/94/50/68/ | 2/0.5/1/ | 50 | N/A |
| | | 94/50/68/94/ | 30/0.5/ | | |
| | | 50/68/50/60 | 1/30/0.5/ | | |
| | | | 1/30/5/ | | |
| | | | 10 | | |
| DH5α-Mediated | 8 | N/A, no | N/A | 0.5 | N/A |
| Assembly | | reaction | | | |
| FastCloning | 9 | 37 | 60 | un- | N/A |
| | | | | specified | |
| Fast-Fusion | 10 | 25 | 15 | un- | N/A |
| Cloning Kit | | | | specified | |
| Fast-Licase | 11 | Vary, but | Vary | 50 | 75 |
| | | <50 since | from | | |
| | | the active | methods | | |
| | | enzyme | | | |
| | | is T4 DNA | | | |
| | | polymerase, | | | |
| | | just | | | |
| | | inactivated | | | |
| | | during rising | | | |
| | | temperature | | | |
| FUSION Seamless | 12 | 37 | 60 | un- | N/A |
| Cloning Kit | | | | specified | |
| GenBuilder DNA | 13, | 50 | 15 | un- | N/A |
| Assembly | 14 | | | specified | |
| GeneArt Seamless | 15 | Room | 30 | un- | N/A |
| Cloning and | | temperature | | specified | |
| Assembly | | | | | |
| Gibson Assembly | 16 | 50 | 15 | 50 | N/A |
| Gibson Assembly | 17 | 37 | 5 | 25 | 75 |
| Ultra | | | | | |
| Homologous | 18 | 37 | 1 | un- | N/A |
| alignment cloning | | | | specified | |
| Hot Fusion | 19 | 50 | 60 | 20 | N/A |
| Hyper Assembly | 20 | Room | 3 | 30-50 | N/A |
| Cloning Kit | | temperature | | | |
| ig-Fusion Cloning | 21 | 50 | 10 | un- | N/A |
| Kit | | | | specified | |
| Improved SLICE | 22 | 22 | 2.5 | 100 | N/A |
| method | | | | | |
| In-Fusion | 23 | 50 | 15 | 50-200 | N/A |
| in vivo E. coli | 24 | N/A, no | N/A | 10 | N/A |
| cloning | | reaction | | | |
| LiClone Fast | 25 | 50 | 5 | un- | N/A |
| Cloning Kits | | | | specified | |
| NEB Hifi builder | 26 | 50 | 15 | 20 | N/A |
| One Step Seamless | 27 | 50 | 15 | 20 | N/A |
| Cloning Mix | | | | | |
| pEASY-Uni | 28 | 50 | 15 | 10 | N/A |
| Seamless | | | | | |
| Cloning and DNA | | | | | |
| Assembly | | | | | |
| Quick and clean | 29 | Room | 10 | un- | N/A |
| cloning | | temperature | | specified | |
| Quick PCR | 30 | 22 | 30 | un- | N/A |
| Cloning Kit | | | | specified | |
| Seamless Cloning | 31 | 45 | 30 | un- | N/A |
| by HEAL | | | | specified | |
| Seamless cloning | 32 | 50 | 30 | 50 | N/A |
| Master Mix (Kit) | | | | | |
| Seamless Ligation | 33 | 37 | 15 | 50-200 | N/A |
| Cloning Extract | | | | | |
| sequence and | 34 | Room | 30 | 150 ng | N/A |
| ligation- | | Temperature | | | |
| independent | | | | | |
| cloning | | | | | |
| Simple enhanced | 35 | 50 | 60 | un- | N/A |
| Gibson Isothermal | | | | specified | |
| Assembly | | | | | |
| simplified DATEL | 36 | 94/94/50/68/ | 2/0.5/1/ | 50 | N/A |
| | | 94/50/68/94/ | 30/0.5/ | | |
| | | 50/68/50/60 | 1/30/0.5/ | | |
| | | | 1/30/5/ | | |
| | | | 10 | | |
| Single 3′- | 37 | 37 | 15 | 1 | N/A |
| exonuclease-based | | | | | |
| multifragment | | | | | |
| DNA | | | | | |
| assembly method | | | | | |
| T5 exonuclease- | 38 | 30 | 40 | 100-200 | N/A |
| dependent | | | | | |
| assembly | | | | | |
| Unnamed SLICE | 39 | 37 | 5 | 10 | N/A |
| method | | | | | |

From Table 1, though there are many variants of molecular cloning methods, for the methods employing only a single reaction step, the temperature of the assembly reaction is no more than 50° C. Inactivation at 75° C. is used in four cases, but is always much higher the actual reaction temperature. The DATEL and simplified DATEL are the exceptions as they require multiple steps, though they are otherwise not much different from the single step procedure; except that they require a different PCR machine procedure, and the actual reaction time is the longest (Table 1), totaling 111.5 min.

Several of the described methods are in vitro recombination systems that assemble and repair overlapping DNA molecules in a single isothermal step. In most of the methods implementing a single reaction step the temperature to generate single-stranded DNA for the homologous region is about 50° C. Most of the available methods require a minimum of 20 ng of plasmid vector to obtain targeted colonies of the transformed 100 bacterial colonies. Thus, the amount of plasmid vector required in the existing method is quite high.

The transformation of the competent cells with the gene insert containing plasmid vector may be mediated by using cations (e.g., Ca²⁺) and low temperature. The efficiency of the competent cells depends upon several factors, including ion concentration and type, treatment time, thermal shock, and incubation time. When transforming a vector into chemically competent Escherichia. coli (E. Coli) bacteria with ampicillin as the drug resistance selection, most commercially available cell transformation procedures allow immediate plating onto agar plates containing ampicillin. Ampicillin hinders cell wall formation but does not promptly kill the cells, giving the bacteria E. Coli sufficient time to synthesize the ampicillin resistance enzyme, preventing destruction by ampicillin in the plate. The secreted enzyme in the media leads to ampicillin degradation, enabling even cells lacking the ampicillin resistance gene to grow. In liquid media, ampicillin resistance enzyme secretions reduce drug resistance pressure, lowering plasmid content and relative protein production. Thus use of the ampicillin as a selective marker may result in transformed cells with low plasmid content.

In contrast, using antibiotics like Kanamycin, Chloramphenicol, Tetracycline, or others that inhibit protein synthesis retains original colonies without generating satellite colonies, as the resistance enzyme is not secreted. Since no drug resistance develops immediately after heat shock, a recovery period is typically required. The competent cells' recovery involves adding 4 to 9-fold SOC media or other recovery media after a 42° C. heat shock, shaking at 37° C. (200 to 300 rpm) for 1 hour, and then spinning down all cells before resuspending a smaller volume for plating. The various methods known in the art require a minimum of 5 minutes to 111.5 minutes (DATEL and simplified DATEL) as the recovery/reaction time for competent cells. Although the SOC media addition and shaking step are not time-consuming for a few samples, they become impractical for high-throughput transformations.

As a result, there has been a need for a method of transformation that can eliminate the requirement of the recovery period after the transformation of competent cells, post uptake of the plasmid vector carrying the foreign gene inserts, and thus significantly reduce the time required for the method. In addition, the method should also be faster, simple in requirement, should have high efficiency, and can adapted to existing PCR machines in terms of system and process parameter requirements.

### SUMMARY

This section provides a general summary of the disclosure and is not a comprehensive disclosure of its full scope or all its features.

Accordingly, the invention relates to a seamless cloning method, comprising a single assembling step (including annealing) of two or more polynucleotides, where one is preferably a plasmid vector and another is a gene insert, conducted between 58° C. and 100° C., where the preferred temperature is the same or greater than one of the following temperatures: 58° C., 59° C., 60° C., 61° C., 62° C., 63° C., 64° C., 65° C., 66° C., 67° C., 68° C., 69° C., 70° C., 71° C., 72° C., 73° C., 74° C., 75° C., 76° C., 77° C., 78° C., 79° C., 80° C., 84° C., 88° C., 92° C., 96° C. and 100° C., and a transformation into chemically competent cells for covalent linking, preferably with a static recovery period. Preferably, the cells in the static recovery period are incubated for 15 minutes from 0° C. to 37° C., followed by cooling to 0-4° C. The competent cells include DH10BC and DH10B and any other cell capable of replicating a plasmid.

In still another embodiment a concentration of the plasmid vector ranges from 0.07-3 ng/kb of the plasmid vector and that of the gene insert ranges from 0.014-9 ng/kb of the gene insert.

In yet another embodiment, the homologous base pair length at 3′- and 5′-end of the plasmid vector and the gene insert is 10-40 base pair and the melting temperature (Tₘ) at both ends of the annealing plasmid and gene insert is in the range of 30-50° C.

In another embodiment, the plasmid vector comprises a selectable marker gene that confers resistance to antibiotics inhibiting protein synthesis. The antibiotics include, but are not limited to, Kanamycin, Chloramphenicol, Tetracycline, and similar compounds, and preferably not an ampicillin-resistant gene.

Additional aspects and advantages of the present disclosure will become apparent to those skilled in this art from the following detailed description, wherein only illustrative embodiments of the present disclosure are shown and described. The present disclosure is capable of other and different embodiments, and several details are capable of modifications in various obvious respects, and its several details are capable of modifications in various obvious respects, all without departing from the disclosure. Accordingly, the descriptions, and examples in this summary are intended for purposes of illustration only and are not intended to limit the scope of the present disclosure.

### DETAILED DESCRIPTION

The embodiments herein and the various features and advantageous details thereof are explained more fully with reference to the non-limiting embodiments that are illustrated in the accompanying drawings and the following description. Numerous variations, changes, and substitutions may occur to those skilled in the art without departing from the invention. It should be understood that various alternatives to the embodiments of the present disclosure herein may be employed.

At the outset, for ease of reference, certain terms used in this application and their meanings as used in this context are set forth. To the extent a term used herein is not defined below, it should be given the broadest definition persons in the pertinent art have given that term as reflected in at least one printed publication or issued patent. Further, the present techniques are not limited by the usage of the terms shown below, as all equivalents, synonyms, new developments, and terms or techniques that serve the same or a similar purpose are considered to be within the scope of the present claims.

The articles “a” and “an” as used herein mean one or more when applied to any feature in embodiments of the present invention described in the specification and claims. The use of “a” and “an” does not limit the meaning to a single feature unless such a limit is specifically stated. The article “the” preceding singular or plural nouns or noun phrases denotes a particular specified feature or particular specified features and may have a singular or plural connotation depending upon the context in which it is used. The adjective “any” means one, some, or all indiscriminately of whatever quantity.

The term “seamless cloning method” is defined as a sequence-independent and scarless insertion of one or more fragments of DNA into a plasmid vector. The method usually employs the Polymerase Chain Reaction (PCR) to amplify the gene of interest, an exonuclease to chew back/remove one strand of the insert and vector ends, and covalently join the insert to the vector through a true phosphodiester bond using a ligase/recombination event, in vivo repair.

The term “Tm” (Melting temperature) is defined as the temperature at which the DNA double helix undergoes denaturation, separating into individual single DNA strands.

The term “transformation” refers to the uptake of the plasmid vector containing a gene insert by a competent cell.

The term “unidirectional exonuclease” is defined as the enzyme having exonuclease activity either from 5-prime to 3-prime or the 3-prime to 5-prime direction. Accordingly, the invention in one aspect relates to a seamless cloning method, comprising a single reaction step of a plasmid vector and a gene insert conducted at between 58° C. and 100° C., but preferably at 67° C.; and a transformation step for chemically competent cells with a static recovery period.

In one embodiment of the present invention, the single reaction step comprises the generation of single-stranded overhang terminal regions of the gene insert that are capable of annealing, and the generation of a linearized vector, wherein the overhanging ends of the plasmid vector and the gene insert are each capable of hybridizing, annealing linearized vector and the gene inert having single-stranded overhangs terminal regions.

In one embodiment of the present invention, the generation of single-stranded overhangs terminal regions of the gene insert is carried out by a unidirectional 3′ to 5′ or a 5′ to 3′ exonuclease.

In yet another embodiment, the present invention relates to a static recovery period for the transformed competent cells such that the cells are incubated for 15 minutes from 0° C. to 37° C., followed by cooling to 0-4° C. The competent cells include DH10BC and DH10B.

In still another embodiment a concentration of the plasmid vector ranges from 0.07-3 ng/kb of the plasmid vector and that of the gene insert ranges from 0.014-9 ng/kb of the gene insert.

In yet another embodiment, the homologous base pair length at the 3′- and 5′-end of the plasmid vector and the gene insert is 10-40 base pair and the melting temperature (Tm) at both ends of the annealing plasmid and gene insert is in the range of 30-50° C.

In another embodiment, the plasmid vector comprises a selectable marker gene that confers resistance to antibiotics inhibiting protein synthesis. The antibiotics include, but are not limited to, Kanamycin, Chloramphenicol, Tetracycline, and similar compounds.
In one embodiment the single-step reaction of a plasmid vector is carried out by Ligation-independent cloning (LIC), Type II Restriction enzyme cloning, the Gibbson assembly method, and the likes thereof.

In another embodiment, seamless cloning is carried out by Ligation-independent cloning (LIC) by amplifying one or more target gene/DNA molecules through the polymerase chain reaction (PCR) using a forward primer and a reverse primer, generating a single-stranded terminal region, annealing DNA fragments with the linearized vector and transforming the competent cells with the annealed vector.

In one embodiment the seamless cloning is carried out by type II restriction endonuclease, such that the plasmid vector and the gene insert are contacted with two or more nucleic acid molecules, each nucleic acid molecule comprising restriction enzyme recognition sites, followed by contacting the plasmid vector and the gene insert with restriction enzyme to generate overhanging ends, excision of a segment of the plasmid vector and the gene insert, to generate a the plasmid vector and the gene insert wherein the overhanging ends of the plasmid vector and the gene insert are each capable of hybridizing. This is followed by the hybridization of overhanging ends and covalent joining of digested nucleic acid molecules to the digested nucleic acid molecule vector to form the recombinant nucleic acid molecule.

In one embodiment, primers with overlapping sequences are designed and used for amplification of the desired inserts using PCR, between the adjacent DNA fragments for their assembly into a cloning vector between the adjacent DNA fragments for their assembly into a cloning vector. The exonuclease is added to create single-stranded 3′ overhangs that facilitate the annealing of fragments sharing complementarity at the overlap region, DNA polymerase is added to fill in gaps within each annealed fragment and DNA ligase to seal nicks in the assembled DNA. The reaction is incubated at 50° C. for carrying out the reaction. The obtained vector carrying the gene of interest is then transformed into the competent cells.

Advantages of the present method include:
1. The method is capable of being carried out in the available PCR machine with no special infrastructure and hardware requirements.
2. The concentration of the plasmid vector and gene insert is very low; e.g., 0.07-3 ng/kb for the plasmid vector and 0.014-9 ng/kb for the gene insert respectively.
3. In the present method, the requirement of the recovery period after the transformation of competent cells is significantly reduced.
4. The present method is suited for high-throughput processing.

### EXAMPLES

### Example 1

### Reaction Temperature Comparison

A linearized vector designated 3701 bp pKBXInH5 having kanamycin resistance selective marker with 12 bp homology region: CAGTCTGGCGGA (SEQ ID NO 7:) . . . TGATAGTCGGCT (SEQ ID NO: 8) was used, The 12 bp homology region is underlined in SEQ ID NO: 1, which shows the full sequence of the linearized vector.

### SEQ ID NO: 1 the Full Sequences of Linearized pKBXI-H5

(SEQ ID NO: 1)
TAATGTGCCTGTCAAATGGACGAAGCAGGGATTCTGCAAACCCTATGCTA
CTCCGTCAAGCCGTCAATTGTCTGATTCGTTACCAATTATGACAACTTGA
CGGCTACATCATTCACTTTTTCTTCACAACCGGCACGGAACTCGCTCGGG
CTGGCCCCGGTGCATTTTTTAAATACCCGCGAGAAATAGAGTTGATCGTC
AAAACCAACATTGCGACCGACGGTGGCGATAGGCATCCGGGTGGTGCTCA
AAAGCAGCTTCGCCTGGCTGATACGTTGGTCCTCGCGCCAGCTTAAGACG
CTAATCCCTAACTGCTGGCGGAAAAGATGTGACAGACGCGACGGCGACAA
GCAAACATGCTGTGCGACGCTGGCGATATCAAAATTGCTGTCTGCCAGGT
GATCGCTGATGTACTGACAAGCCTCGCGTACCCGATTATCCATCGGTGGA
TGGAGCGACTCGTTAATCGCTTCCATGCGCCGCAGTAACAATTGCTCAAG
CAGATTTATCGCCAGCAGCTCCGAATAGCGCCCTTCCCCTTGCCCGGCGT
TAATGATTTGCCCAAACAGGTCGCTGAAATGCGGCTGGTGCGCTTCATCC
GGGCGAAAGAACCCCGTATTGGCAAATATTGACGGCCAGTTAAGCCATTC
ATGCCAGTAGGCGCGCGGACGAAAGTAAACCCACTGGTGATACCATTCGC
GAGCCTCCGGATGACGACCGTAGTGATGAATCTCTCCTGGCGGGAACAGC
AAAATATCACCCGGTCGGCAAACAAATTCTCGTCCCTGATTTTTCACCAC
CCCCTGACCGCGAATGGTGAGATTGAGAATATAACCTTTCATTCCCAGCG
GTCGGTCGATAAAAAAATCGAGATAACCGTTGGCCTCAATCGGCGTTAAA
CCCGCCACCAGATGGGCATTAAACGAGTATCCCGGCAGCAGGGGATCATT
TTGCGCTTCAGCCATACTTTTCATACTCCCGCCATTCAGAGAAGAAACCA
ATTGTCCATATTGCATCAGACATTGCCGTCACTGCGTCTTTTACTGGCTC
TTCTCGCTAACCAAACCGGTAACCCCGCTTATTAAAAGCATTCTGTAACA
AAGCGGGACCAAAGCCATGACAAAAACGCGTAACAAAAGTGTCTATAATC
ACGGCAGAAAAGTCCACATTGATTATTTGCACGGCGTCACACTTTGCTAT
GCCATAGCATTTTTATCCATAAGATTAGCGGATCCTACCTGACGCTTTTT
ATCGCAACTCTCTACTGTTTCTCCATACCCGTTTTTTTGGCATATGGAGC
TCCCATGGTGTACACCTAGGAGATCTGCGATCGCGTTTGGAGGTAATAAA
TGGCAGTGCAACACTCTAATGCGCCTCTGATCGATTGTGGAGCCGAAATG
AAAAAACAGCATAAGGAGGCCGCGCCTGAAGGTGCTGCACCTGCTCAAGG
GAAAGCTCCTGCGGCTGAAGCGAAAAAAGAAGAAGCGCCCAAACCCAAAC
GGCTCGTCTCTAGTGGGATCGAGGAAAACCTTTATTTCCAGTCTCATCAT
CATCACCACCACGGCTCT
CAGTCTGGCGGAxxxxxxxxxxxxTGATAGTCGGCTG
CAACTTTATCCGCCTGTGACTAGTGCTAGCGGCGCGCCCTCGAGGGTACC
GAATTCGCGGCCGCCGGTCTGATAAAACAGAATTTGCCTGGCGGCAGTAG
CGCGGTGGTCCCACCTGACCCCATGCCGAACTCAGAAGTGAAACGCCGTA
GCGCCGATGGTAGTGTGGGTTCTCCCCATGCGAGAGTAGGGAACTGCCAG
GCATCAAATAAAACGAAAGGCTCAGTCGAAAGACTGGGCCTTTCGTTTTA
TCTGTTGTTTGTCGGTGAACGCTCTCCTGAGTAGGACAAATCCGCCGGGA
GCGGATTTGAACGTTGCGAAGCAACGGCCCGGAGGGTGGCGGGCAGGACG
CCCGCCATAAACTGCCAGGCATCAAATTAAGCAGAAGGCCATCCTGACGG
ATGGCCTTTTTGCGTTTCTACAAACTCTTTTGTTTATTTTTCTAAATACA
TTCAAATATGTATCCGCTCATGAGACAATAACCCTGATAAATGCTTCAAT
AATATTGAAAAAGGAAGAGTATGAGCCATATTCAACGGGAAACGTCTTGC
TCGAGGCCGCGATTAAATTCCAACATGGATGCTGATTTATATGGGTATAA
ATGGGCTCGCGATAATGTCGGGCAATCAGGTGCGACAATCTATCGATTGT
ATGGGAAGCCCGATGCGCCAGAGTTGTTTCTGAAACATGGCAAAGGTAGC
GTTGCCAATGATGTTACAGATGAGATGGTCAGACTAAACTGGCTGACGGA
ATTTATGCCTCTTCCGACCATCAAGCATTTTATCCGTACTCCTGATGATG
CATGGTTACTCACCACTGCGATCCCCGGGAAAACAGCATTCCAGGTATTA
GAAGAATATCCTGATTCAGGTGAAAATATTGTTGATGCGCTGGCAGTGTT
CCTGCGCCGGTTGCATTCGATTCCTGTTTGTAATTGTCCTTTTAACAGCG
ATCGCGTATTTCGTCTCGCTCAGGCGCAATCACGAATGAATAACGGTTTG
GTTGATGCGAGTGATTTTGATGACGAGCGTAATGGCTGGCCTGTTGAACA
AGTCTGGAAAGAAATGCATAAGCTTTTGCCATTCTCACCGGATTCAGTCG
TCACTCATGGTGATTTCTCACTTGATAACCTTATTTTTGACGAGGGGAAA
TTAATAGGTTGTATTGATGTTGGACGAGTCGGAATCGCAGACCGATACCA
GGATCTTGCCATCCTATGGAACTGCCTCGGTGAGTTTTCTCCTTCATTAC
AGAAACGGCTTTTTCAAAAATATGGTATTGATAATCCTGATATGAATAAA
TTGCAGTTTCATTTGATGCTCGATGAGTTTTTCTAATAAAAGGATCTAGG
TGAAGATCCTTTTTGATAATCTCATGACCAAAATCCCTTAACGTGAGTTT
TCGTTCCACTGAGCGTCAGACCCCGTAGAAAAGATCAAAGGATCTTCTTG
AGATCCTTTTTTTCTGCGCGTAATCTGCTGCTTGCAAACAAAAAAACCAC
CGCTACCAGCGGTGGTTTGTTTGCCGGATCAAGAGCTACCAACTCTTTTT
CCGAAGGTAACTGGCTTCAGCAGAGCGCAGATACCAAATACTGTCCTTCT
AGTGTAGCCGTAGTTAGGCCACCACTTCAAGAACTCTGTAGCACCGCCTA
CATACCTCGCTCTGCTAATCCTGTTACCAGTGGCTGCTGCCAGTGGCGAT
AAGTCGTGTCTTACCGGGTTGGACTCAAGACGATAGTTACCGGATAAGGC
GCAGCGGTCGGGCTGAACGGGGGGTTCGTGCACACAGCCCAGCTTGGAGC
GAACGACCTACACCGAACTGAGATACCTACAGCGTGAGCTATGAGAAAGC
GCCACGCTTCCCGAAGGGAGAAAGGCGGACAGGTATCCGGTAAGCGGCAG
GGTCGGAACAGGAGAGCGCACGAGGGAGCTTCCAGGGGGAAACGCCTGGT
ATCTTTATAGTCCTGTCGGGTTTCGCCACCTCTGACTTGAGCGTCGATTT
TTGTGATGCTCGTCAGGGGGGCGGAGCCTATGGAAAAACGCCAGCAACGC
GGCCTTTTTACGGTTCCTGGCCTTTTGCTGGCCTTTTGCTCACATG

The insert (xxxxxxxxxxxx) in SEQ ID NO: 1 is a 741 bp enhanced Green Fluorescent Protein (eGFP) gene fragment, which has the same homologous region as above, CAGTCTGGCGGA (SEQ ID NO: 7) . . . TGATAGTCGGCT (SEQ ID NO: 8) on the gene.

The insert is a gene fragment without an adaptor. All gene fragments in this application are synthesized by Twist Bioscience, CA. The full sequence of the insert is shown in SEQ ID NO: 2:

### SEQ ID NO: 2: The Insert Sequence of eGFP

CAGTCTGGCGGAATGGTGAGCAAGGGCGAGGAGCTGTTCACC
GGGGTGGTGCCCATCCTGGTCGAGCTGGACGGCGACGTAAAC
GGCCACAAGTTCAGCGTGTCCGGCGAGGGCGAGGGCGATGCC
ACCTACGGCAAGCTGACCCTGAAGTTCATCTGCACCACCGGC
AAGCTGCCCGTGCCCTGGCCCACCCTCGTGACCACCCTGACC
TACGGCGTGCAGTGCTTCAGCCGCTACCCCGACCACATGAAG
CAGCACGACTTCTTCAAGTCCGCCATGCCCGAAGGCTACGTC
CAGGAGCGCACCATCTTCTTCAAGGACGACGGCAACTACAAG
ACCCGCGCCGAGGTGAAGTTCGAGGGCGACACCCTGGTGAAC
CGCATCGAGCTGAAGGGCATCGACTTCAAGGAGGACGGCAAC
ATCCTGGGGCACAAGCTGGAGTACAACTACAACAGCCACAAC
GTCTATATCATGGCCGACAAGCAGAAGAACGGCATCAAGGTG
AACTTCAAGATCCGCCACAACATCGAGGACGGCAGCGTGCAG
CTCGCCGACCACTACCAGCAGAACACCCCCATCGGCGACGGC
CCCGTGCTGCTGCCCGACAACCACTACCTGAGCACCCAGTCC
GCCCTGAGCAAAGACCCCAACGAGAAGCGCGATCACATGGTC
CTGCTGGAGTTCGTGACCGCCGCCGGGATCACTCTCGGCATG
GACGAGCTGTACAAGTGATAGTCGGCT

Briefly, the reaction comparison procedure is as follows: 1 μl of 11.1 ng/μl linearized pKBInH5 (3 ng for 1 kb vector), 1 μl 6.67 ng/μl eGFP fragment (9 ng for 1 kb insert), 1 μl of 3×ZY cloning master mix (Zycloning, Woburn, MA) were mixed. The PCR machine (T100, Biorad) was set to 4° C. for an indefinite time. After the temperature of 4° C., was attained, a PCR tube containing the reaction mixture was loaded in the machine. The 4° C. setting was reset to a higher reaction temperature as shown in Table 2 for 30 seconds, and reset to 4° C. and held indefinitely. 50 μl of competent cells DH10BC (Zycloning, Woburn, MA) were added to the reaction mixture, and the temperature was reset again at 42° C. to heat shock the sample for one minute. The sample was held at 4° C. for 15 minutes, followed by 4° C. for an indefinite time. All transformed mixture was then plated on a Kanamycin (50 μg/ml) containing-agar plate and incubated at overnight 37° C. The results are shown in Table 1.

TABLE 1
Reaction temperature comparison

| Reaction Temp ° C. | Colony Number |
|---|---|
| 50 | 1 |
| 51 | 4 |
| 52 | 3 |
| 53 | 0 |
| 54 | 1 |
| 55 | 7 |
| 56 | 0 |
| 57 | 5 |
| 58 | 12 |
| 59 | 20 |
| 60 | 23 |
| 61 | 27 |
| 62 | 58 |
| 63 | 79 |
| 64 | 89 |
| 65 | 95 |
| 66 | 100 |
| 67 | 141 |
| 68 | 68 |
| 69 | 83 |
| 70 | 122 |
| 71 | 192 |
| 72 | 103 |
| 73 | 195 |
| 74 | 269 |
| 75 | 251 |
| 76 | 97 |
| 77 | 169 |
| 78 | 132 |
| 79 | 65 |
| 80 | 218 |
| 84 | 194 |
| 88 | 193 |
| 92 | 93 |
| 96 | 101 |
| 100 | 86 |

The result shows that the colony number varies below the reaction temperature of 57° C. However, once the reaction time was kept at 58° C. and above, the colony number increased significantly. With an increase in the reaction temperature up to 100° C., an increase in the colony number was observed. In theory, double-stranded DNA starts to denature at temperatures over 90° C. However, the PCR machine has a ramp-up and ramp-down speed, and the ramp period is enough for the reaction to proceed sufficiently. This high reaction temperature used in the current invention is novel and inventive when compared with other currently available cloning methods that employ a single reaction step.

After multiple comparison rounds and standardization, 67° C. was selected as the standard reaction temperature. The results show that there is no significant reaction when the temperature is less than 50° C. Further, no significant difference was observed when the reaction was carried out at room temperature. In addition, the order of addition of sample, vector, insert, and 3× ZY cloning master mix was observed to be non-significant since the greater part of the reaction takes place at high temperatures.

In this example, as noted above, the vector was used at 3 ng for every kb plasmid vector, and the gene insert was used at 9 ng for every kb of the gene insert which is significantly lower than in most of the other methods currently known (50-100 ng/kb). The available commercial kits generally recommend higher concentrations than published papers. In practice, however, protocols for this method require much higher amounts Although the recommendation may be 0.5 ng of the vector plasmid, but actually requires 100 ng for no more than 50 colonies (DH5alpha mediated assembly).

The reaction time was standardized at 30 seconds, which is faster than most available methods.

### Example 2

### Reaction Time Comparison

The vector and insert in this section used in the experiments were the same as previously standardized (Example 1). The competent cells used were from a different batch. The procedure employed a PCR machine, which involved a ramp-up and ramp-down time. The PCR machine was set at 67° C., and the PCR tube contained the same reaction mix as used in Example 1. The exact time of the incubation of the sample at each temperature may be a little shorter than the tested time. The colony number achieved in this procedure is shown in Table 2. With the right frame insert, the eGFP gives green fluorescence to show the correct insert ratio.

TABLE 2

| Reaction time | | Green | Correct |
|---|---|---|---|
| on 67° C. | Colony | Fluorescent | ratio |
| plate (sec) | number | Colony number | % |
| 1 | 1 | 0 | N/A |
| 2 | 14 | 5 | 35.7 |
| 4 | 6 | 0 | N/A |
| 8 | 13 | 4 | 30.8 |
| 15 | 31 | 22 | 71.0 |
| 30 | 312 | 303 | 97.1 |

The colony number obtained/observed after 30 seconds of reaction time was 312, which is enough for most molecular biology applications. A reaction time of 15 seconds results in a significantly higher colony number than the background. However, an additional 15 seconds increase in the reaction time, increases the colonies about 10-fold, as shown. When the correct insert ratio is also considered, the 15-second reaction time gives a much lower correct ratio (71.0%) when compared to the 30-second reaction where it increases to 97.1%. The 30-second reaction produces good results in terms of both colony number and insert ratio, and therefore, the 30-second reaction was used as the standard reaction time.

### Example 3

### Static Recovery Time Comparison for Kanamycin Plates

The vector used in this example is pKLpositive vector, a 2328 bp kanamycin vector as in SEQ ID NO: 3:

### SEQ ID NO: 3: pKLpositive Vector

CAGGTGGCACTTTTCGGGGAAATGTGCGCGGAACCCCTATTTGTTTATTT
TTCTAAATACATTCAAATATGTATCCGCTCATGAGACAATAACCCTGATA
AATGCTTCAATAATATTGAAAAAGGAAGAGTATGAGCCATATTCAACGGG
AAACGTCTTGCTCGAGGCCGCGATTAAATTCCAACATGGATGCTGATTTA
TATGGGTATAAATGGGCTCGCGATAATGTCGGGCAATCAGGTGCGACAAT
CTATCGATTGTATGGGAAGCCCGATGCGCCAGAGTTGTTTCTGAAACATG
GCAAAGGTAGCGTTGCCAATGATGTTACAGATGAGATGGTCAGACTAAAC
TGGCTGACGGAATTTATGCCTCTTCCGACCATCAAGCATTTTATCCGTAC
TCCTGATGATGCATGGTTACTCACCACTGCGATCCCCGGGAAAACAGCAT
TCCAGGTATTAGAAGAATATCCTGATTCAGGTGAAAATATTGTTGATGCG
CTGGCAGTGTTCCTGCGCCGGTTGCATTCGATTCCTGTTTGTAATTGTCC
TTTTAACAGCGATCGCGTATTTCGTCTCGCTCAGGCGCAATCACGAATGA
ATAACGGTTTGGTTGATGCGAGTGATTTTGATGACGAGCGTAATGGCTGG
CCTGTTGAACAAGTCTGGAAAGAAATGCATAAGCTTTTGCCATTCTCACC
GGATTCAGTCGTCACTCATGGTGATTTCTCACTTGATAACCTTATTTTTG
ACGAGGGGAAATTAATAGGTTGTATTGATGTTGGACGAGTCGGAATCGCA
GACCGATACCAGGATCTTGCCATCCTATGGAACTGCCTCGGTGAGTTTTC
TCCTTCATTACAGAAACGGCTTTTTCAAAAATATGGTATTGATAATCCTG
ATATGAATAAATTGCAGTTTCATTTGATGCTCGATGAGTTTTTCTAATAA
AAGGATCTAGGTGAAGATCCTTTTTGATAATCTCATGACCAAAATCCCTT
AACGTGAGTTTTCGTTCCACTGAGCGTCAGACCCCGTAGAAAAGATCAAA
GGATCTTCTTGAGATCCTTTTTTTCTGCGCGTAATCTGCTGCTTGCAAAC
AAAAAAACCACCGCTACCAGCGGTGGTTTGTTTGCCGGATCAAGAGCTAC
CAACTCTTTTTCCGAAGGTAACTGGCTTCAGCAGAGCGCAGATACCAAAT
ACTGTCCTTCTAGTGTAGCCGTAGTTAGGCCACCACTTCAAGAACTCTGT
AGCACCGCCTACATACCTCGCTCTGCTAATCCTGTTACCAGTGGCTGCTG
CCAGTGGCGATAAGTCGTGTCTTACCGGGTTGGACTCAAGACGATAGTTA
CCGGATAAGGCGCAGCGGTCGGGCTGAACGGGGGGTTCGTGCACACAGCC
CAGCTTGGAGCGAACGACCTACACCGAACTGAGATACCTACAGCGTGAGC
TATGAGAAAGCGCCACGCTTCCCGAAGGGAGAAAGGCGGACAGGTATCCG
GTAAGCGGCAGGGTCGGAACAGGAGAGCGCACGAGGGAGCTTCCAGGGGG
AAACGCCTGGTATCTTTATAGTCCTGTCGGGTTTCGCCACCTCTGACTTG
AGCGTCGATTTTTGTGATGCTCGTCAGGGGGGCGGAGCCTATGGAAAAAC
GCCAGCAACGCGGCCTTTTTACGGTTCCTGGCCTTTTGCTGGCCTTTTGC
TCACATGCTGGCACGACAGGTTTCCCGACTGGAAAGCGGGCAGTGAGCGC
AACGCAATTAATGTGAGTTAGCTCACTCATTAGGCACCCCAGGCTTTACA
CTTTATGCTTCCGGCTCGTATGTTGTGTGGAATTGTGAGCGGATAACAAT
TTCACACAGGAAACAGCTATGACCATGATTACGCCCATATGGAGCTCCCA
TGGTGTACACCTAGGAGATCTGCGATCGCGTTTGGAGGTAAATAAATGGG
TCATCACCATCATCATCACGGAAGCGGTTCTAGCGGTATGGTGAGCAGTG
GCGAAGATATTTTCTCGGGCTTGGTTCCGATTCTGATCGAGCTGGAGGGC
GATGTGAACGGTCATCGTTTTAGCGTTCGCGGTGAAGGTTATGGCGACGC
GAGCAACGGCAAACTGGAAATTAA
GTTCATCTGCACGAxxxxxxxxxxxxACGACCAATAGCGT
GCTGAGCAAAGATCCGCAGGAACGCCGTGATCACATGGTCCTGGTGGAAT
TTGTGACCGCTGCGGGCTTGAGCCTGGGTATGGACGAGCTGTATAAGAGC
TAAGTGACTAGTGCTGTGACTAGTGCTAGCGGCGCGCCCTCGAGGGTACC
GAATTCGCGGCCGC

The GTTCATCTGCACGA (SEQ ID NO. 9) xxxxxxxxxxxxACGACCAATAGCGT (SEQ ID NO. 10) contains the homologous region, which contains a part of fuGFP (40). The remaining fuGFP is the insert in SEQ ID NO: 4.

### SEQ ID NO: 4 Remaining fuGFP as 483 bp Insert

GTTCATCTGCACGACCGGTCGCCTGCCGGTGCCTTGGCCGAC
CTTGGTGACGACCTTGTCGTATGGCGTGCAGTGTTTTGCGAA
GTATCCGGAGCACATGCGCCAAAACGATTTCTTTAAAAGTGC
GATGCCGGACGGTTACGTCCAGGAGCGTACCATTTCCTTCAA
GGAAGATGGCACGTACAAAACTCGCGCAGAGGTTAAGTTTGA
AGGTGAAGCGCTGGTCAATCGTATCGATTTGAAGGGTTTGGA
GTTTAAAGAGGATGGTAACATTCTGGGCCATAAACTGGAGTA
TAGCTTCAACAGCCATTATGTTTACATTACGGCAGACAAGAA
TCGTAACGGCTTGGAGGCCCAATTCCGTATTCGCCACAATGT
TGATGACGGTAGCGTCCAACTGGCCGACCATTACCAACAGAA
CACCCCAATTGGTGAGGGTCCGGTGTTGCTGCCGGAACAACA
CTATCTGACGACCAATAGCGT

A mixture of 6.984 ng/μl pKL positive vector and 1.449 ng/μl fuGFP insert was taken as the ZY cloning positive control for the vector. In order to insert a molar ratio of 1:1, 2 μl of ZY cloning positive control (ZyCloning, Woburn, MA) and 1.449 ng/μl fuGFP insert were mixed with 1 μl of 3× ZY Cloning master mix (ZyCloning, Woburn, MA) and the reaction was performed at 67° C. for 30 secs. 50 μl of chemically competent cells DH10BC (ZyCloning, Woburn, MA) was added to the reaction mixture. The cells were exposed to 42° C. heat shock, a separate cell transformation mixture was incubated at 37° C. for a set time, without adding any media and without shaking, followed by 15 minutes on ice for control. After the incubation time, the cells were plated on Kanamycin plates and incubated at 37° C. overnight. The results are shown in Table 3:

TABLE 3
Static incubation time effect comparison

| Static incubation time | Colony numbers |
|---|---|
| 37° C. 30 seconds | 10 |
| 37° C. 1 minute | 13 |
| 37° C. 2 min | 57 |
| 37° C. 4 min | 50 |
| 37° C. 8 min | 103 |
| 0° C. 15 min | 139 |

The incubation at 37° C. did not speed up the reaction time, as seen by the 8-minute incubation at 37° C. yielding lower colony numbers than 0° C. for 15 min. At 1 minute incubation or less, the colony number is even lower. In a 2-minute incubation at 37° C., the colony numbers obtained were only 41% compared to 15 mins. at 0° C. At 8 min, the colony numbers increased to 74% of that obtained at 15 mins at 0° C. Thus, the standard procedure was standardized at a low temperature for 15 min. The lowest temperature that can be set on a PCR machine is 4° C., so 4° C. for 15 min. was standardized as the static incubation parameters. In this transformation procedure, there is no waiting time after competent cells are mixed with the reaction sample. The present method has a distinct advantage over the conventional method that uses kanamycin, chloramphenicol, tetracycline, and other antibiotics as selective markers. The method of the present invention has a shorter recovery time than the conventional methods that employ, media addition and shaking procedure, which are critical for the conventional method of direct transformation. From reaction to plating, this method is one of the fastest methods for molecular cloning.

### Example 4

### Different Competent Cell Comparisons

A total of 7 competent cells commercially available and supplied by various vendors, were compared to the DH10B (ZyCloning, Woburn, MA), wherein in one batch the fast recovery step of 15 minutes at 4° C. was carried out, and in another batch, the conventional steps of addition of 9× recovery media (New England Biolabs, Ipswich, MA) and shaking incubation at 37° C. for 1 hour was employed, all the cells were spun down. All cells were plated on a kanamycin (50 μg/ml) plate and incubated at 37° C. The results are shown in Table 4.

TABLE 4
The comparison of DH10B from different sources in different recovery modes

| | | | | Colony | Colony Number | |
|---|---|---|---|---|---|---|
| | | | | Number | obtained from | Fold |
| | | | | obtained | 9x recovery | increased |
| | | | | from static | media, followed | from static |
| | | | | incubation at | by incubation | incubation to |
| Competent | | | Claimed | 4° C. for 15 | at 37° C. for | conventional |
| Cell | Supplier | Reference | efficiency | minutes | one hour | step |
| E. cloni | Biosearchtech | 41 | 1 × 10⁹ | 335 | 10000+ | 30+ |
| 10G | | | | | | |
| DH10B | GoldBio | 42 | 8.2 × 10⁶ | 90 | 297 | 3.3 |
| Ig 10B | Intact Genomics | 43 | 1.0 × 10¹⁰ | 1 | 1075 | 1075 |
| 10-beta | New England | 44 | 1-3 × 10⁹ | 17 | 10000++ | 588+ |
| | Biolabs | | | | | |
| DH10β | Origene | 45 | 1 × 10⁸ | 2 | 194 | 97 |
| DH10B | Thermo Fisher | 46 | 1 × 10⁹ | 0 | 637 | infinity |
| Mix & Go | ZymoResearch | 47 | 1 × 10⁸-10⁹ | 1 | 1059 | 1059 |
| 10B | | | | | | |
| DH10B | ZyCloning | This | Normal | 609 | 1446 | 2.4 |
| | | application | method | | | |

The competent cells from ZyCloning work better than any other competent cells as presented in Table 4 when the static recovery mode is used. The E. coli 10G cells (Biosearchtech) achieved 55% colony numbers of the reference cells DH10B(ZyCloning) and DH10B cells (Goldbio) achieved 15% colony numbers of the reference cells. When the recovery media from New England Biolabs was used, the competent cells from Biosearchtech and New England Biolabs worked much better. The DH10B cells from ZyCloning increased about 2.4 fold after incubation and shaking with recovery media.

In the case of complicated nucleotide assembly having more than three polynucleotide pieces assembly, the colony outcome can be increased by additional shaking with recovery media. It must be noted that standardization for maximum efficiency with different media can be carried out to recover the maximum number of colonies with other competent cells. In the comparison carried out in Table 4, the DH10B competent cells from the method of the present invention have their competency around 1×10⁸ when the normal recovery method is used, and around 4×10⁷ when the static recovery method is applied. The competent cells of the present invention can be named “FastRecovery” competent cells.

If the best competent cells in the shaking with recovery media are used, to achieve a 100 colonies goal, the reaction vector and insert can be as low as 0.070 ng and 0.014 ng, which is lower than any of the claimed lower limits in any of the conventional methods.

### Example 5

### Homology Length Checking

In the examples, the length of the homology bases is 12/12 bp (Examples 1 and 2) and 14/14 bp (Examples 3 and 4), which is already lower than the conventionally used 15 bp limit. The vector used in this example is pKLShv, a plasmid with Kanamycin resistance and a portion of sfGFP(48) as SEQ ID NO:5.

### SEQ ID NO:5: The Sequence of pKLShv

CAGGTGGCACTTTTCGGGGAAATGTGCGCGGAACCCCTATTTG
TTTATTTTTCTAAATACATTCAAATATGTATCCGCTCATGAGA
CAATAACCCTGATAAATGCTTCAATAATATTGAAAAAGGAAGA
GTATGAGCCATATTCAACGGGAAACGTCTTGCTCGAGGCCGCG
ATTAAATTCCAACATGGATGCTGATTTATATGGGTATAAATGG
GCTCGCGATAATGTCGGGCAATCAGGTGCGACAATCTATCGAT
TGTATGGGAAGCCCGATGCGCCAGAGTTGTTTCTGAAACATGG
CAAAGGTAGCGTTGCCAATGATGTTACAGATGAGATGGTCAGA
CTAAACTGGCTGACGGAATTTATGCCTCTTCCGACCATCAAGC
ATTTTATCCGTACTCCTGATGATGCATGGTTACTCACCACTGC
GATCCCCGGGAAAACAGCATTCCAGGTATTAGAAGAATATCCT
GATTCAGGTGAAAATATTGTTGATGCGCTGGCAGTGTTCCTGC
GCCGGTTGCATTCGATTCCTGTTTGTAATTGTCCTTTTAACAG
CGATCGCGTATTTCGTCTCGCTCAGGCGCAATCACGAATGAAT
AACGGTTTGGTTGATGCGAGTGATTTTGATGACGAGCGTAATG
GCTGGCCTGTTGAACAAGTCTGGAAAGAAATGCATAAGCTTTT
GCCATTCTCACCGGATTCAGTCGTCACTCATGGTGATTTCTCA
CTTGATAACCTTATTTTTGACGAGGGGAAATTAATAGGTTGTA
TTGATGTTGGACGAGTCGGAATCGCAGACCGATACCAGGATCT
TGCCATCCTATGGAACTGCCTCGGTGAGTTTTCTCCTTCATTA
CAGAAACGGCTTTTTCAAAAATATGGTATTGATAATCCTGATA
TGAATAAATTGCAGTTTCATTTGATGCTCGATGAGTTTTTCTA
ATAAAAGGATCTAGGTGAAGATCCTTTTTGATAATCTCATGAC
CAAAATCCCTTAACGTGAGTTTTCGTTCCACTGAGCGTCAGAC
CCCGTAGAAAAGATCAAAGGATCTTCTTGAGATCCTTTTTTTC
TGCGCGTAATCTGCTGCTTGCAAACAAAAAAACCACCGCTACC
AGCGGTGGTTTGTTTGCCGGATCAAGAGCTACCAACTCTTTTT
CCGAAGGTAACTGGCTTCAGCAGAGCGCAGATACCAAATACTG
TCCTTCTAGTGTAGCCGTAGTTAGGCCACCACTTCAAGAACTC
TGTAGCACCGCCTACATACCTCGCTCTGCTAATCCTGTTACCA
GTGGCTGCTGCCAGTGGCGATAAGTCGTGTCTTACCGGGTTGG
ACTCAAGACGATAGTTACCGGATAAGGCGCAGCGGTCGGGCTG
AACGGGGGGTTCGTGCACACAGCCCAGCTTGGAGCGAACGACC
TACACCGAACTGAGATACCTACAGCGTGAGCTATGAGAAAGCG
CCACGCTTCCCGAAGGGAGAAAGGCGGACAGGTATCCGGTAAG
CGGCAGGGTCGGAACAGGAGAGCGCACGAGGGAGCTTCCAGGG
GGAAACGCCTGGTATCTTTATAGTCCTGTCGGGTTTCGCCACC
TCTGACTTGAGCGTCGATTTTTGTGATGCTCGTCAGGGGGGCG
GAGCCTATGGAAAAACGCCAGCAACGCGGCCTTTTTACGGTTC
CTGGCCTTTTGCTGGCCTTTTGCTCACATGCTGGCACGACAGG
TTTCCCGACTGGAAAGCGGGCAGTGAGCGCAACGCAATTAATG
TGAGTTAGCTCACTCATTAGGCACCCCAGGCTTTACACTTTAT
GCTTCCGGCTCGTATGTTGTGTGGAATTGTGAGCGGATAACAA
TTTCACACAGGAAACAGCTATGACCATGATTACGCCCATATGG
AGCTCCCATGGTGTACACCTAGGAGATCTGCGATCGCGTTTGC
GATCGCGTTTGGAGGTAAATAAATGGGTCATCACCATCATCAT
CACGGAAGCGGTTCTAGCGGTATGGTCAGTAAAGGTGAGGAGT
TGTTTACTGGTGTCGTTCCTATTCTTGTCGAGTTGGACGGGGA
TGTGAACGGGCACAAGTTTTCGGTACGTGGCGAGGGTGAAGGG
GATGCAACTAATGGCAAGTTGACGTTGAAGTTTATATGCACGA
CTGGGAAGCTCCCAGTGCCATGGCCAACTTTGGTGACTACCTT
AxxxxxxxxxxxxCACTATCTTTCTACACAAAGCCTGCTTTCT
AAGGACCCAAATGAGAAACGCGACCACATGGTCTTGCTTGAGT
TCGTGACAGCGGCAGGGATTACCTTGGGCATGGACGAGCTCTA
CAAGGGGATCGAGGAAAACCTGTACTTCTAAGTGACTAGTGCT
GTGACTAGTGCTAGCGGCGCGCCCTCGAGGGTACCGAATTCGC
GGCCGC

The xxxxxxxxxxxx in SEQ ID NO:5 is part of the plasmid vector where the gene insert is inserted. The gene insert is the remaining part of the Superfolder Green Fluorescent Protein (sfGFP), shown in SEQ ID NO:6, plus the additional sequence at both ends of the vector region.

### SEQ ID NO:6: The Remaining Part of sfGFP (Gene Insert)

ACCTACGGTGTACAATGCTTCTCGCGTTACCCCGATCACATGAAGCAACACGATT TCTTCAAGTCAGCAATGCCTGAAGGTTACGTCCAAGAACGTACTATATCATTCAA AGACGACGGTACCTACAAGACTCGGGCGGAAGTTAAGTTCGAAGGTGACACTTT AGTCAATCGTATCGAGTTAAAGGGTATCGATTTCAAAGAGGATGGCAACATTTTA GGACACAAGCTGGAGTACAACTTTAACAGCCACAATGTATACATTACTGCCGAC AAGCAAAAGAACGGCATCAAGGCAAATTTCAAGATTAGACATAACGTCGAAGAC GGCTCCGTGCAATTAGCAGATCATTATCAACAGAACACGCCGATCGGCGACGGC CCCGTGTTATTACCCGACAAT

The additional homologous sequence on the 5′ and 3′ ends is shown in Table 6.
The Tm (melting temperature for denaturation of DNA) was calculated by the given formula
Tm=(wA+xT)*2+(yG+zC)*4 for short sequences that are less than 13 bp, and
Tm for sequences longer than 14 bp was calculated by the given formula

Tm=64.9+41*(yG+zC−16.4)/(wA+xT+yG+zC)(49),

wherein, w, x, y, z are the numbers of A, T, G, and C in the sequence.

TABLE 6
Homologous sequence length and Cloning efficiency

| Homo- | | | | | |
|---|---|---|---|---|---|
| logous | | Sim- | | Sim- | Insert |
| length | | ple | | ple | amount |
| BP | Sequence on 5′ | Tm | Sequence on 3′ | Tm | (ng) |
| 0 | N/A | 0 | N/A | 0 | 3.618 |
| 1 | A | 2 | C | 4 | 3.636 |
| 2 | TA | 4 | CA | 6 | 3.654 |
| 3 | TTA | 6 | CAC | 10 | 3.672 |
| 4 | CTTA | 10 | CACT | 12 | 3.69 |
| 5 | CCTTA | 14 | CACTA | 14 | 3.708 |
| 6 | ACCTTA | 16 | CACTAT | 16 | 3.726 |
| 7 | TACCTTA | 18 | CACTATC | 20 | 3.744 |
| 8 | CTACCTTA | 22 | CACTATCT | 22 | 3.762 |
| 9 | ACTACCTTA | 24 | CACTATCTT | 24 | 3.78 |
| 10 | GACTACCTTA | 26 | CACTATCTTT | 26 | 3.798 |
| | (SEQ ID NO: 11) | | (SEQ ID NO: 12) | | |
| 11 | TGACTAC | 28 | CACTATC | 30 | 3.816 |
| | CTTA | | TTTC | | |
| | (SEQ ID NO: 13) | | (SEQ ID NO: 14) | | |
| 12 | GTGACTA | 32 | CACTATC | 32 | 3.834 |
| | CCTTA | | TTTCT | | |
| | (SEQ ID NO: 15) | | (SEQ ID NO: 16) | | |
| 13 | GGTGAC | 36 | CACTATC | 34 | 3.852 |
| | TACCTTA | | TTTCTA | | |
| | (SEQ ID NO: 17) | | (SEQ ID NO: 18) | | |
| 14 | TGGTGACTAC | 34.4 | CACTATCTTT | 31.5 | 3.87 |
| | CTTA | | CTAC | | |
| | (SEQ ID NO: 19) | | (SEQ ID NO: 20) | | |
| 15 | TTGGTGACT | 36.5 | CACTATCTT | 33.7 | 3.888 |
| | ACCTTA | | TCTACA | | |
| | (SEQ ID NO: 21) | | (SEQ ID NO: 22) | | |
| 16 | TTTGGTGACT | 38.3 | CACTATCTT | 38.3 | 3.906 |
| | ACCTTA | | TCTACAC | | |
| | (SEQ ID NO: 23) | | (SEQ ID NO: 24) | | |
| 17 | CTTTGGTGAC | 42.2 | CACTATCTT | 39.8 | 3.924 |
| | TACCTTA | | TCTACACA | | |
| | (SEQ ID NO: 25) | | (SEQ ID NO: 26) | | |
| 18 | ACTTTGGTGA | 43.5 | CACTATCTT | 41.2 | 3.942 |
| | CTACCTTA | | TCTACACAA | | |
| | (SEQ ID NO: 27) | | (SEQ ID NO: 28) | | |
| 19 | AACTTTG | 44.6 | CACTAT | 42.5 | 3.96 |
| | GTGACTA | | CTTTCTAC | | |
| | CCTTA | | ACAAA | | |
| | (SEQ ID NO: 29) | | (SEQ ID NO: 30) | | |
| 20 | CAACTTTGG | 47.7 | CACTATCT | 45.6 | 3.978 |
| | TGACTAC | | TTCTACA | | |
| | CTTA | | CAAAG | | |
| | (SEQ ID NO: 31) | | (SEQ ID NO: 32) | | |
| 21 | CCAACTTT | 50.5 | CACTATCTT | 48.5 | 3.996 |
| | GGTGACTA | | TCTACAC | | |
| | CCTTA | | AAAGC | | |
| | (SEQ ID NO: 33) | | (SEQ ID NO: 34) | | |
| 22 | GCCAACTT | 53 | CACTATCTT | 51.1 | 4.014 |
| | TGGTGACT | | TCTACAC | | |
| | ACCTTA | | AAAGCC | | |
| | (SEQ ID NO: 35) | | (SEQ ID NO: 36) | | |
| 23 | GGCCAACTT | 55.3 | CACTATCT | 51.7 | 4.032 |
| | TGGTGACT | | TTCTACACA | | |
| | ACCTTA | | AAGCCT | | |
| | (SEQ ID NO: 37) | | (SEQ ID NO: 38) | | |
| 24 | TGGCCAACTT | 55.7 | CACTATCTT | 54 | 4.05 |
| | TGGTGACT | | TCTACACA | | |
| | ACCTTA | | AAGCCTG | | |
| | (SEQ ID NO: 39) | | (SEQ ID NO: 40) | | |
| 25 | ATGGCCAACT | 56 | CACTATCTT | 56 | 4.068 |
| | TTGGTGACT | | TCTACACAA | | |
| | ACCTTA | | AGCCTGC | | |
| | (SEQ ID NO: 41) | | (SEQ ID NO: 42) | | |
| 26 | CATGGCCAAC | 58 | CACTATCTT | 56.4 | 4.086 |
| | TTTGGTGAC | | TCTACACAA | | |
| | TACCTTA | | AGCCTGCT | | |
| | (SEQ ID NO: 43) | | (SEQ ID NO: 44) | | |
| 27 | CCATGGCC | 59.7 | CACTATCT | 56.7 | 4.104 |
| | AACTTTG | | TTCTACAC | | |
| | GTGACTA | | AAAGC | | |
| | CCTTA | | CTGCTT | | |
| | (SEQ ID NO: 45) | | (SEQ ID NO: 46) | | |
| 28 | GCCATGGCCA | 61.4 | CACTATCTTT | 57 | 4.122 |
| | ACTTTGGTGA | | CTACACAAA | | |
| | CTACCTTA | | GCCTGCTTT | | |
| | (SEQ ID NO: 47) | | (SEQ ID NO: 48) | | |
| 29 | TGCCATGGCC | 61.5 | CACTATCTTTC | 58.7 | 4.14 |
| | AACTTTGGTG | | TACACAAAG | | |
| | ACTACCTTA | | CCTGCTTTC | | |
| | (SEQ ID NO: 49) | | (SEQ ID NO: 50) | | |
| 30 | GTGCCATG | 63 | CACTATCT | 58.9 | 4.158 |
| | GCCAACTTT | | TTCTACAC | | |
| | GGTGACTA | | AAAGCCT | | |
| | CCTTA | | GCTTTCT | | |
| | (SEQ ID NO: 51) | | (SEQ ID NO: 52) | | |
| 31 | AGTGCCATG | 63 | CACTATCT | 59.1 | 4.176 |
| | GCCAACTT | | TTCTACAC | | |
| | TGGTGAC | | AAAGCCT | | |
| | TACCTTA | | GCTTTCTA | | |
| | (SEQ ID NO: 53) | | (SEQ ID NO: 54) | | |
| 32 | CAGTGCCA | 64.4 | CACTATCTT | 59.3 | 4.194 |
| | TGGCCAACT | | TCTACACAA | | |
| | TTGGTGAC | | AGCCTGCT | | |
| | TACCTTA | | TTCTAA | | |
| | (SEQ ID NO: 55) | | (SEQ ID NO: 56) | | |
| 33 | CCAGTGCC | 65.6 | CACTATCTT | 60.7 | 4.212 |
| | ATGGCCAA | | TCTACACA | | |
| | CTTTGGTGA | | AAGCCTGCT | | |
| | CTACCTTA | | TTCTAAG | | |
| | (SEQ ID NO: 57) | | (SEQ ID NO: 58) | | |
| 34 | CCCAGTGC | 66.8 | CACTATCTT | 62 | 4.23 |
| | CATGGCCA | | TCTACACA | | |
| | ACTTTGGTG | | AAGCCTGCT | | |
| | ACTACCTTA | | TTCTAAGG | | |
| | (SEQ ID NO: 59) | | (SEQ ID NO: 60) | | |
| 35 | TCCCAGTGCC | 66.8 | CACTATCTT | 62.1 | 4.248 |
| | ATGGCCAA | | TCTACACAA | | |
| | CTTTGGTGA | | AGCCTGCTT | | |
| | CTACCTTA | | TCTAAGGA | | |
| | (SEQ ID NO: 61) | | (SEQ ID NO: 62) | | |
| 36 | CTCCCAGT | 67.9 | CACTATCTT | 63.3 | 4.266 |
| | GCCATGGC | | TCTACACAA | | |
| | CAACTTTGGT | | AGCCTGCTT | | |
| | GACTACCTTA | | TCTAAGGAC | | |
| | (SEQ ID NO: 63) | | (SEQ ID NO: 64) | | |
| 37 | GCTCCCAGTG | 68.9 | CACTATCTT | 64.5 | 4.284 |
| | CCATGGCC | | TCTACACAA | | |
| | AACTTTGGT | | AGCCTGCTT | | |
| | GACTACCTTA | | TCTAAGGACC | | |
| | (SEQ ID NO: 65) | | (SEQ ID NO: 66) | | |
| 38 | AGCTCCC | 68.8 | CACTATC | 65.5 | 4.302 |
| | AGTGCC | | TTTCTAC | | |
| | ATGGCCAA | | ACAAAGCC | | |
| | CTTTGGTG | | TGCTTTCT | | |
| | ACTACCTTA | | AAGGACCC | | |
| | (SEQ ID NO: 67) | | (SEQ ID NO: 68) | | |
| 39 | AAGCTCCC | 68.7 | CACTATC | 65.5 | 4.32 |
| | AGTGCCATG | | TTTCTAC | | |
| | GCCAACT | | ACAAAGCC | | |
| | TTGGTGAC | | TGCTTTCTA | | |
| | TACCTTA | | AGGACCCA | | |
| | (SEQ ID NO: 69) | | (SEQ ID NO: 70) | | |
| 40 | GAAGCTCC | 69.6 | CACTATCT | 65.5 | 4.338 |
| | CAGTGCCA | | TTCTAC | | |
| | TGGCCAA | | ACAAAGCC | | |
| | CTTTGGTGA | | TGCTTTCTA | | |
| | CTACCTTA | | AGGACCCAA | | |
| | (SEQ ID NO: 71) | | (SEQ ID NO: 72) | | |

1 μl of 7.2 ng/μl linearized pKLshv, 1 μl of insert length×0.009 ng/μl, as in Table 7, and 1 μl of 3× ZY Cloning master mix (ZyCloning, Woburn, MA) were mixed and heated at 67° C. for 30 seconds. 50 μl DH10BC (ZyCloning, Woburn, MA) was added to the reaction mix, and the cells were heat-shocked on a PCR machine at 42° C. for one minute, followed by incubation at 4° C. for 15 minutes. The cells were plated on Kanamycin (50 g/ml) containing plates and the plates were incubated at 37° C. overnight. The green fluorescent colonies having the correct orientation of the eGFP protein were counted and the ratio was calculated. The results are shown in Table 7.

TABLE 7
homologous sequence length and cloning efficiency

| | | | | Proper | |
|---|---|---|---|---|---|
| Homo- | 5′ | 3′ | | inserted | Correct |
| logous | Homo- | Homo- | | colonies | ratio of |
| length | logous | logous | Total | with Green | insertion |
| BP | Tm | Tm | Colonies | Fluorescence | % |
| 0 | 0 | 0 | 0 | 0 | N/A |
| 1 | 2 | 4 | 0 | 0 | 0 |
| 2 | 4 | 6 | 1 | 0 | 0 |
| 3 | 6 | 10 | 1 | 0 | 0 |
| 4 | 10 | 12 | 3 | 0 | 0 |
| 5 | 14 | 14 | 0 | 0 | N/A |
| 6 | 16 | 16 | 0 | 0 | N/A |
| 7 | 18 | 20 | 3 | 0 | 0 |
| 8 | 22 | 22 | 0 | 0 | N/A |
| 9 | 24 | 24 | 4 | 1 | 25 |
| 10 | 26 | 26 | 16 | 13 | 81.3 |
| 11 | 28 | 30 | 51 | 41 | 80.4 |
| 12 | 32 | 32 | 18 | 16 | 88.9 |
| 13 | 36 | 34 | 14 | 11 | 78.6 |
| 14 | 34.4 | 31.5 | 205 | 193 | 94.5 |
| 15 | 36.5 | 33.7 | 361 | 343 | 95.0 |
| 16 | 38.3 | 38.3 | 251 | 240 | 95.6 |
| 17 | 42.2 | 39.8 | 139 | 125 | 90.0 |
| 18 | 43.5 | 41.2 | 223 | 209 | 93.7 |
| 19 | 44.6 | 42.5 | 360 | 346 | 96.1 |
| 20 | 47.7 | 45.6 | 288 | 275 | 95.4 |
| 21 | 50.5 | 48.5 | 164 | 154 | 93.9 |
| 22 | 53 | 51.1 | 76 | 69 | 90.8 |
| 23 | 55.3 | 51.7 | 258 | 249 | 96.5 |
| 24 | 55.7 | 54 | 131 | 127 | 97.0 |
| 25 | 56 | 56 | 113 | 106 | 93.8 |
| 26 | 58 | 56.4 | 122 | 120 | 98.4 |
| 27 | 59.7 | 56.7 | 109 | 106 | 97.3 |
| 28 | 61.4 | 57 | 74 | 69 | 93.3 |
| 29 | 61.5 | 58.7 | 240 | 237 | 98.8 |
| 30 | 63 | 58.9 | 106 | 105 | 99.1 |
| 31 | 63 | 59.1 | 65 | 63 | 96.9 |
| 32 | 64.4 | 59.3 | 77 | 77 | 100 |
| 33 | 65.6 | 60.7 | 185 | 180 | 97.3 |
| 34 | 66.8 | 62 | 318 | 313 | 98.4 |
| 35 | 66.8 | 62.1 | 304 | 288 | 94.7 |
| 36 | 67.9 | 63.3 | 145 | 143 | 98.6 |
| 37 | 68.9 | 64.5 | 148 | 146 | 98.6 |
| 38 | 68.8 | 65.5 | 120 | 116 | 96.7 |
| 39 | 68.7 | 65.5 | 59 | 57 | 96.6 |
| 40 | 69.6 | 65.5 | 114 | 106 | 93.0 |

For DNA fragments with lengths of 8 base pairs (bp) or less and a melting temperature (Tm) less than or equal to 22/22, no instances of gene inserts with the correct orientation were observed. Fragments with lengths between 9 and 13 bp, featuring Tm values of 24/24 and 36/34, respectively, yielded colony numbers that, while significant, were relatively low. Elevating the Tm to 36/36 using the short calculation method resulted in a substantial increase in colony numbers, with a generally consistent positive rate exceeding 90% for base pair lengths up to 40 bp. Based on the experimental evidence, the Tm was standardized to be at least 36° C. for both ends.

Sequences with high repeat ratios, self-complementarity, very high or low GC percentages, or two homologous sequences that are highly similar are generally unsuitable selections for the homologous sequence in the present method. Experimental verification indicated that DNA fragments obtained from restriction enzyme digestion, gene synthesis, PCR, high-performance liquid chromatography (HPLC), or gel-purified primers, particularly a 12 bp fragment with a Tm of 36° C., yielded favourable results. However, if the PCR primer is crude, longer homologous sequences are generally required due to the inherent reliability challenges associated with primers synthesized from the 3′ end, which tend to be less than 100% reliable at the 5′ end.

In summary, the novel ZY Cloning System method encompasses homologous sequence design, vector, and insert preparation. The reaction typically involves a mixture of 1 μl of vector, 1 μl of insert, and 1 μl of 3× ZY cloning master mix, conducted at a temperature of 67° C. for 30 seconds, followed by a 50 μl competent cell transformation. Fast static incubation recovery is employed for drug resistance, other than Ampicillin, after heat shock The foregoing description of the specific embodiments will so fully reveal the general nature of the embodiments herein that others can, by applying current knowledge, readily modify and/or adapt for various applications such specific embodiments without departing from the generic concept, and, therefore, such adaptations and modifications should and are intended to be comprehended within the meaning and range of equivalents of the disclosed embodiments. It is to be understood that the phraseology or terminology employed herein is for the purpose of description and not of limitation. Therefore, while the embodiments herein have been described in terms of preferred embodiments, those skilled in the art will recognize that the embodiments herein can be practiced with modification within the spirit and scope of the appended claims.

### REFERENCES

1. https://eng.bioneer.com/20-k-7110-cfg.html
2. https://www.mclab.com/Choo-Choo-Cloning-Kits.html
3. https://www.tamar.co.il/rna-dna-magnetic-clean-beads-4/
4. https://www.lubio.ch/applications/molecular-cloning/ultra-rapid-cloning
5. https://web.archive.org/web/20170711224046/https://www.systembio.com/molecular-tools/cold-fusion-cloning/overview/
6. Sun, S. et al. (2015) Biotechnol Biotechnol Equip. 29(1): 105-110.
7. Jing, P. et al. (2016) ACS Synth. Biol. 2016, 5, 9, 1028-1032
8. Kostylev, M. et al., (2015) PLOS ONE, https://doi.org/10.1371/journal.pone.0137466
9. Li, C. et al. (2011), BMC Biotechnology, 11, Article number, 92
10. https://www.genecopoeia.com/product/fast-fusion-cloning-kit/
11. http://www.abdesignlabs.com/technical-resources/licase-protocol/
12. https://www.realgenelabs.com/fusion-seamless-cloning-kit-514000
13. https://www.genscript.com/genbuilder-dna-assembly.html
14. https://www.thermofisher.com/us/en/home/life-science/cloning/seamless-cloning-and-genetic-assembly/geneart-seamless-cloning-and-assembly.html
15. Gibson, D. G., et al. (2009) Nat. Methods 6, 343-345.
16. Gibson, D. G., et al. (2010) Science 329, 52-56.
17. Chen, C. et al. (2018) Biotechniques. 60, NO. 3S
18. Tan, L., et al. (2018), PeerJ 6:e5146; DOI 10.7717/peerj.5146
19. Fu, C. et al. (2014) PLOS ONE, https://doi.org/10.1371/journal.pone.0115318
20. https://www.apexbt.com/hyperfusiontm-high-fidelity-dna-polymerase.html
21. https://intactgenomics.com/product/ig-fusion-cloning-kit/
22. Jeong, J Y. et al. (2012) Applied and Environmental Microbiology Vol. 78, No. 15
23. https://www.takarabio.com/products/cloning/in-fusion-seamless-cloning/in-fusion-snap-assembly-for-high-throughput-workflows?gclid=CjwKCAiA2fmdBhBpEiwA4CcHzTGdiilMdqvXqQwwS7aW8dT tWAjfdXMHCGDAOVIrDdPWn7ZxXaaJuhoCISkQAvD_BWE
24. Nozaki, S. et al. (2019) J Bacteriol. 2019 Mar. 1; 201(5): e00660-18.
25. https://www.lifesct.com/Cloning-Kits
26. https://www.neb.com/applications/cloning-and-synthetic-biology/dna-assembly-and-cloning/nebuilder-hifi-dna-assembly
27. https://www.cwbiosciences.com/goods/index/id/63
28. https://www.civicbio.com/product/peasy-uni-seamless-cloning-and-assembly-kit/
29. Thieme, F., et al., (2014) Methods Mol Biol, 1116:37-48. doi: 10.1007/978-1-62703-764-8_3.
30. https://bpsbioscience.com/fast-simple-and-efficient-cloning-kit-without-competent-cells
31. https://lifeome.com/seamless-cloning-by-healtrade-p-584453.html?osCsid-6898fe79621bbf9c59fcbd73d694c2bd
32. https://www.biobasic.com/seamless-cloning-master-mix-kit-4108
33. Zhang, Y., (2014), Methods Mol Biol. 2014; 1116: 235-244.
34. Li, M. et al. (2007) Nature Methods volume 4, pages 251-256
35. Rabe, B. (2020) https://www.biorxiv.org/content/10.1101/2020.06.14.150979v1
36. Ding, W. (2017) Bioengineered. 2017; 8(3): 296-301.
37. Dao, V L. et al. (2022) Scientific Reports volume 12, Article number: 4004 20
38. Xia, Y. et al. (2018) Nucleic Acids Research, Volume 47, Issue 3, e15
39. Motohashi. K., (2015) BMC Biotechnology, 15, Article number 47

What is claimed is:

1. A cloning method, wherein the method comprises:
assembling in a single step, two or more polynucleotides, wherein the assembly reaction is conducted at from 58° C. to 100° C.; and
transforming the assembled product into a competent cell for covalently linking of the polynucleotides.

2. The cloning method according to claim 1, wherein the assembly reaction temperature is the same or greater than a particular one of the following temperatures: 58° C., 59° C., 60° C., 61° C., 62° C., 63° C., 64° C., 65° C., 66° C., 67° C., 68° C., 69° C., 70° C., 71° C., 72° C., 73° C., 74° C., 75° C., 76° C., 77° C., 78° C., 79° C., 80° C., 84° C., 88° C., 92° C., 96° C. and 100° C.

3. The cloning method of claim 1 wherein at least one of the polynucleotides is a plasmid vector.

4. The cloning method of claim 1 wherein the transformation step includes a static recovery period.

5. The cloning method according to claim 4, wherein the static recovery period comprises incubating the transformed competent cells for 15 minutes at a temperature of 0° C. to 37° C.

6. The cloning method according to claim 1, wherein the competent cells are selected from a group comprising DH10BC and DH10B.

7. The cloning method according to claim 1, wherein the single step assembly reaction comprises;
a. providing single-stranded overhangs terminal regions of a polynucleotide that are capable of annealing;
b. providing a linearized plasmid vector, wherein the overhanging ends of the plasmid vector and the polynucleotide are each capable of hybridizing; and
c. annealing the linearized vector and the polynucleotide having single-stranded overhangs terminal regions.

8. The cloning method according to claim 3, wherein the plasmid vector and the polynucleotide comprise homologous base pair length at 3′- and 5′-end of 10-40 base pair length.

9. The cloning method according to claim 3, wherein a melting temperature (Tm) at both ends of the linearized plasmid vector and polynucleotide annealing is in the range of 30-50° C.

10. The cloning method according to claim 1, wherein the generation of single-stranded overhangs terminal regions of the polynucleotide is carried out by a unidirectional 3′ to 5′ or a 5′ to 3′ exonuclease enzyme.

11. The cloning method according to claim 3, wherein the concentration of the plasmid vector ranges from 0.07-3 ng/kb the plasmid vector.

12. The cloning method according to claim 1, wherein the concentration of the polynucleotide ranges from 0.014-9 ng/kb of the polynucleotide.

13. The cloning method according to claim 3, wherein the plasmid vector comprises a selectable marker gene that confers resistance to antibiotics inhibiting protein synthesis.

14. The cloning method according to claim 13, wherein the antibiotics are selected from a group comprising Kanamycin, Chloramphenicol, and Tetracycline.

15. The cloning method according to claim 3, wherein the plasmid vector comprises a selectable marker gene that is not an ampicillin-resistant gene.

16. The cloning method according to claim 3, wherein the plasmid vector sequence is selected from a group comprising SEQ ID NO: 1, SEQ ID NO:3, and SEQ ID NO:5.

17. The cloning method according to claim 5 further including incubation at a temperature of 0-4° C.
